(19) United States
(12) Patent Application Publication
(10) Pub. No.: US 2008/0119467 A1
(43) Pub. Date: May 22, 2008

(54) Purine Derivatives, Compositions Containing Them and Use Thereof

(75) Inventors: Chantal CARREZ, Thiais (FR); Florence FASSY, Paris (FR); Patrick MAILLIET, Fontenay Sous Bois (FR); Fabienne THOMPSON, Paris (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMA S.A., Antony (FR)

(21) Appl. No.: 11/773,572

(22) Filed: Jul. 5, 2007

Related U.S. Application Data
(63) Continuation of application No. PCT/FR06/00065, filed on Jan. 11, 2006.

(30) Foreign Application Priority Data
Jan. 13, 2005 (FR) 0500349

(51) Int. Cl. A61K 31/52; A61K 31/5377; A61P 35/00; C07D 487/04

(52) U.S. Cl. 514/234.2; 514/252.16; 514/263.3; 514/263.4; 544/277; 544/118

(57) ABSTRACT

Purine derivatives of formula (IA1) or (IB1), compositions containing them and use thereof as medicinal products, in particular in oncology, are described

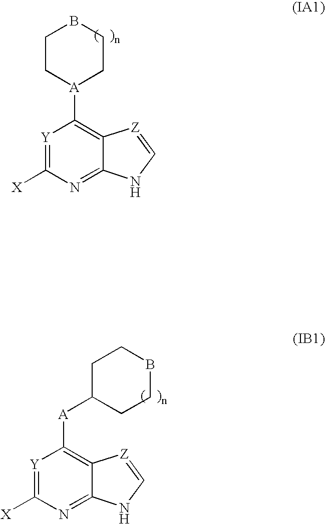

The present invention relates to novel chemical compounds, in particular novel purine derivatives, compositions containing them, and their use as medicinal products.

More particularly, according to a first aspect, the invention relates to novel purine derivatives displaying anti-cancer activity, and in particular inhibitory activity against the Hsp90 chaperone protein, and more particularly via inhibition of the ATPase-type catalytic activity of the Hsp90 chaperone protein.

The present invention thus relates to the products corresponding to the following formula (IA1) or (IB1):

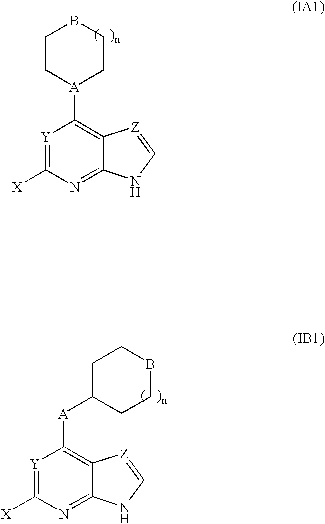

in which Y and Z, which may be identical or different, represent N or CH, it being understood that at least one of Y and Z represents N,
1) X represents a halogen atom;
2) When the general formula is (IA1), A represents N or CH;
3) When the general formula is (IB1), A represents O, S, NH, CH2 or CHR;
4) B represents O, S, NR′, CH2 or CHR;
5) R represents a hydrogen atom; or a C1-C3 alkyl radical
6) R′ represents a hydrogen atom; or a C1-C7 alkyl radical; or a C2-C7 alkenyl or alkynyl radical; or a (CH2)ₙ-aryl or heteroaryl radical; a —CH(aryl)₂ radical; or a C(Z1)-aryl or heteroaryl radical; the aryl or heteroaryl rings, mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms and the group comprising alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, in which the alkyl portions can be C1-C3.
7) n=0, 1, 2
8) Z1 represents an oxygen or sulfur atom or a radical NR′, with R′ as defined previously,
   said products of formula (IA1) or (IB1) being in all possible isomeric forms: racemic, enantiomeric and diastereoisomeric, as well as salts of addition with mineral and organic acids or with inorganic and organic bases of said products of formula (IA1) or (IB1),
   provided that the product of formula (IA1) is not one of the following compounds:

| CAS number | Structure |
|---|---|
| 4010-81-5 | 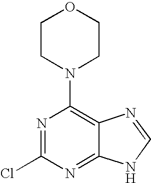 |
| 4854-10-8 | 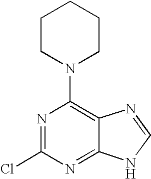 |
| 121371-16-2 | 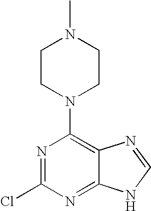 |
| 135394-21-7 | 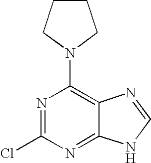 |
| 362524-68-3 | 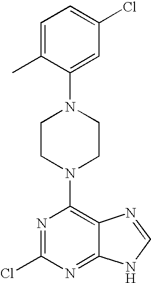 |

and that the product of formula (IB1) is not one of the following compounds:

| CAS number | Structure |
|---|---|
| 439803-59-5 | 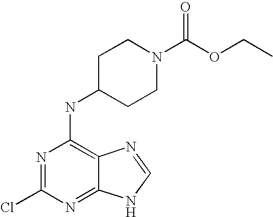 |
| 237422-23-0 | 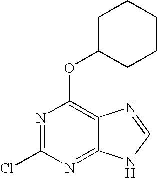 |
| 204633-47-6 | 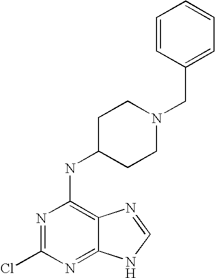 |
| 39639-45-7 | 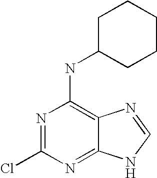 |

The present invention thus relates to the products of formula (IA1) as defined above, characterized in that said products correspond to the following formula (IA), (IIA) or (IIIA):

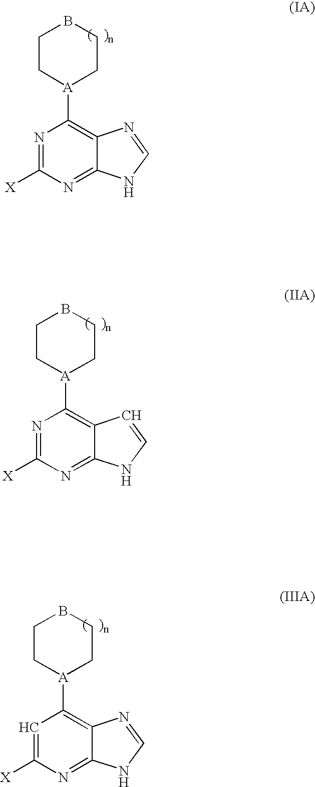

in which:
1) X represents a halogen atom;
2) A represents N or CH;
3) B represents O, S, NR′, CH2 or CHR′;
4) R represents a hydrogen atom; or a C1-C3 alkyl radical
5) R′ represents a hydrogen atom; or a C1-C7 alkyl radical; or a C2-C7 alkenyl or alkynyl radical; or a (CH2)ₙ-aryl or heteroaryl radical; a —CH(aryl)2 radical; or a C(Z1)-aryl or heteroaryl radical; the aryl or heteroaryl rings, mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms and the group comprising alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, the alkyl portions of which can be C1-C3.
6) n=0, 1, 2
7) Z1 represents an oxygen or sulfur atom or a radical NR′, with R′ as defined previously,
   said products of formula (IA1) being in all possible isomeric forms: racemic, enantiomeric and diastereoisomeric, as well as salts of addition with mineral and organic acids or with inorganic and organic bases of said products of formula (IA1),

The present invention thus relates to the products of formula (IB1) as defined above, characterized in that said products correspond to the following formula (IB), (IIB) or (IIIB):

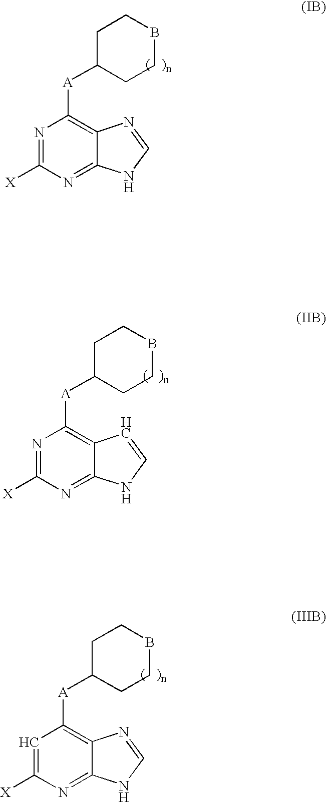

in which:
1) X represents a halogen atom;
2) A represents O, S, NH, CH2 or CHR;
3) B represents O, S, NR′, CH2 or CHR′;
4) R represents a hydrogen atom; or a C1-C3 alkyl radical
5) R′ represents a hydrogen atom; or a C1-C7 alkyl radical; or a C2-C7 alkenyl or alkynyl radical; or a (CH2)ₙ-aryl or heteroaryl radical; a —CH(aryl)2 radical; or a C(Z1)-aryl or heteroaryl radical; the aryl or heteroaryl rings, mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms and the group comprising alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, the alkyl portions of which can be C1-C3.
6) n=0, 1, 2
7) Z1 represents an oxygen or sulfur atom or a radical NR′, with R′ as defined previously,
   said products of formula (IB1) being in all possible isomeric forms: racemic, enantiomeric and diastereoisomeric, as well as salts of addition with mineral and organic acids or with inorganic and organic bases of said products of formula (IB1),

The purine derivatives under consideration here correspond to the following general formulas (IA) or (IB):

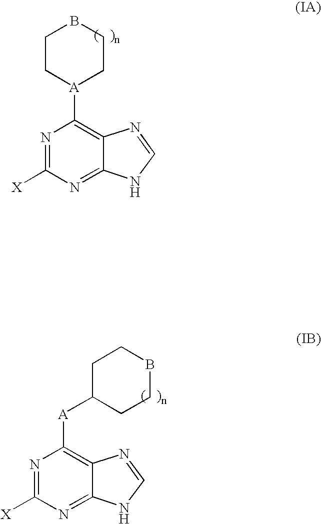

Products corresponding to general formula (IA) and in which A=N, X═Cl are known:

| CAS number | Structure |
|---|---|
| 4010-81-5 | 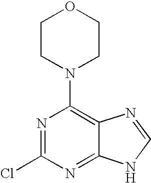 |
| 4854-10-8 | 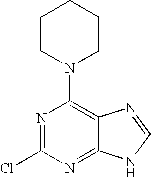 |
| 121371-16-2 | 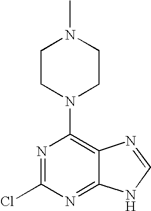 |
| 135394-21-7 | 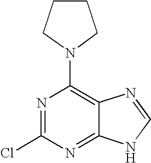 |
| 362524-68-3 | 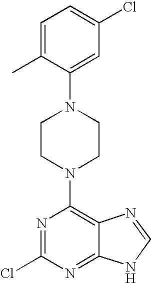 |

Products corresponding to general formula (IB) and in which A=O or NH, and X═Cl are known:

| CAS number | Structure |
|---|---|
| 439803-59-5 | 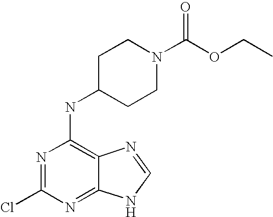 |
| 39639-45-7 | 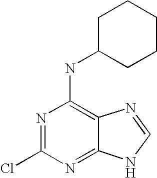 |
| 237422-23-0 | 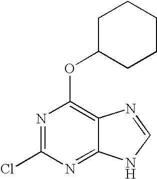 |
| 204633-47-6 | 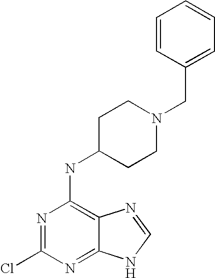 |

Patent application EP300726 claims piperazine derivatives of purines as hypoglycemic agents. The products claimed cannot be substituted with a halogen atom in position 6 of the purine ring.

Patent application WO04/035740 claims amino-morpholinopurine derivatives, which can be used for treating pathologies associated with overproduction of interleukin IL 12. The products claimed have a substituent of type X-Ar(Het) in position 8 of the purine ring.

Patent application WO02/051843 claims a method of preparation of purine derivatives as well as the use thereof as antifungal agents. These derivatives have a substituent of type NH(R^{y}) in position 6 of the purine ring.

Now, surprisingly, it has been found that products corresponding to the following general formula (IA) or (IB) display considerable inhibitory activity on the Hsp90 chaperone:

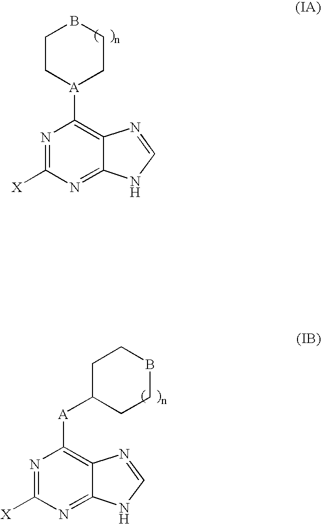

in which:
1) X represents a halogen atom;
2) When the general formula is (IA), A represents N or CH;
3) When the general formula is (IB), A represents O, S, NH, CH2 or CHR;
4) B represents O, S, NR′, CH2 or CHR′;
5) R represents a hydrogen atom; or a C1-C3 alkyl radical
6) R′ represents a hydrogen atom; or a C1-C7 alkyl radical; or a C2-C7 alkenyl or alkynyl radical; or a (CH2)ₙ-aryl or heteroaryl radical; or a C(Z1)-aryl or heteroaryl radical; the aryl or heteroaryl rings, mono- or bicyclic with 5 to 10 ring members, can contain from 0 to 3 heteroatoms, which may be identical or different, selected from O, S or N, and can optionally be substituted with one or more halogen atoms or with one or more radicals selected from the group comprising alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, O—C(O)N(alkyl)₂, the alkyl portions of which can be C1-C3.
7) n=0, 1, 2
8) Z1 represents an oxygen or sulfur atom or a radical NR′, with R′ as defined previously,
   in racemic form, enriched in one enantiomer, enriched in one diastereoisomer, tautomers thereof, prodrugs thereof and pharmaceutically acceptable salts thereof, provided that the product of formula (IA) is not one of the following compounds:

| CAS number | Structure |
|---|---|
| 4010-81-5 | 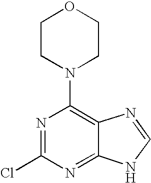 |
| 4854-10-8 | 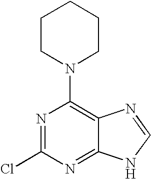 |
| 121371-16-2 | 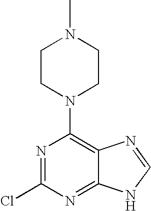 |
| 135394-21-7 | 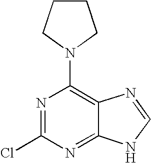 |
| 362524-68-3 | 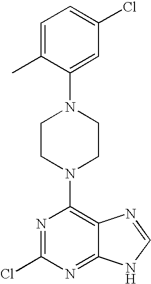 |

and that the product of formula (IB) is not one of the following compounds:

| CAS number | Structure |
|---|---|
| 439803-59-5 | 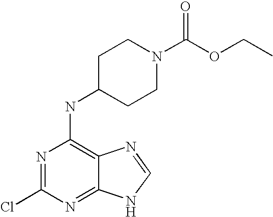 |
| 39639-45-7 | 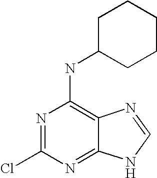 |
| 237422-23-0 | 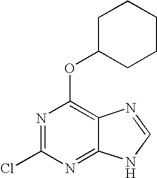 |
| 204633-47-6 | 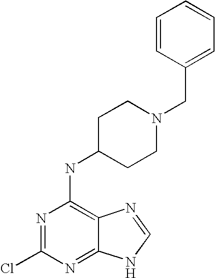 |

As an example of halogen atom that X can represent, we may mention chlorine (Cl), fluorine, bromine, or iodine.

Products of general formula (IA) or (IB) for which X═Cl are preferred.

As examples of mono- or bicyclic, aryl and heteroaryl rings with 5 to 10 ring members and which can contain from 0 to 3 heteroatoms, which may be identical or different, selected from O, S or N, which may optionally be substituted, we may mention the phenyl, pyridyl, pyrimidine, triazine, pyrrolyl, imidazolyl, thiazolyl, pyrrazolyl, furyl, thienyl, indolyl, indazolyl, azaindolyl, azaindazolyl, isobenzofuranyl, isobenzothienyl, benzoxazolyl, benzothiazolyl, quinoleyl, isoquinoleyl, cinnolyl, quinazolyl, naphthyridyl, triazolyl or tetrazolyl groups.

Products of general formula (IA) for which A=N are preferred.

Products of general formula (IA) for which A=N, B=NR′ or CHR′ are preferred.

Products of general formula (IA) for which A=N, B=NR′ or CHR′, and n=1 are preferred.

The present invention notably relates to the products as defined above, characterized in that R′ is selected from the group comprising phenyl, phenylmethyl, phenylamino, phenylcarbonyl, pyridyl, pyrimidinyl or quinoleinyl, the phenyl and pyridyl radicals being optionally substituted with one or more radicals selected from halogen atoms and the alkyl, hydroxyalkyl, Oalkyl, CF3 and CONH2 radicals.

When B is NR′ or CHR′, a preferred substituent R′ can be selected from phenyl, phenyl substituted with at least one radical selected from halogen atom, Oalkyl, —C(O)NH2, or phenylmethyl, or phenylamino, or pyridyl, or pyrimidinyl or quinoleinyl.

More preferably, among the products of general formula (IA), those for which X═Cl, A=N, and n=1 are preferred.

It is also preferable to choose, from the products of formula (IB), those for which A=NH.

Products of general formula (IB) for which A=NH, B=CH2 are preferred.

Products of general formula (IB) for which X═Cl, A=NH are preferred.

Thus, among the products of formulas (IA1) or (IB1) as defined above, we may mention the compounds with the following names:
2-chloro-6-[4-(phenylmethyl)-piperazin-1-yl]-1H-purine monohydrochloride
2-chloro-6-[4-(pyridin-2-yl)-piperazin-1-yl]-1H-purine
2-chloro-6-(morpholin-4-yl)-1H-purine
2-chloro-6-(thiamorpholin-4-yl)-1H-purine
2-chloro-6-(piperidin-1-yl)-1H-purine
2-chloro-6-[4-(diphenylmethyl)-piperazin-1-yl]1H-purine
2-chloro-6-[4-(phenylmethyl)-piperidin-1-yl]-1H-purine monohydrochloride
2-chloro-6-(4-phenyl-piperazin-1-yl)-1H-purine monohydrochloride
2-chloro-6-[4-(pyridin-3-yl)-piperazin-1-yl]1H-purine
2-chloro-6-[4-(3-carboxamido-phenyl)-piperazin-1-yl]1H-purine
2-chloro-6-[4-(phenyl)carbonyl-piperazin-1-yl]1H-purine
2-chloro-6-[4-(phenylamino)-piperidin-1-yl]-1H-purine monohydrochloride
2-chloro-6-[4-(phenylmethyl)-piperidin-1-yl]-1H-purine monohydrochloride
2-chloro-6-[4-(6-methyl-pyridin-2-yl)-piperazin-1-yl]-1H-purine monohydrochloride
2-chloro-6-[4-(pyrimidin-2-yl)-piperazin-1-yl]-1H-purine monohydrochloride
2-chloro-6-[4-(quinolein-2-yl)-piperazin-1-yl]-1H-purine monohydrochloride
2-chloro-6-[4-(2-trifluoromethyl-phenyl)-piperazin-1-yl]-1H-purine
2-fluoro-[4-(pyridin-2-yl)-piperazin-1-yl]-1H-purine
2-[4-(5-chloro-3H-imidazo[4,5-b]pyridin-7-yl)-piperazin-1-yl]-benzamide
{2-[4-(2-chloro-9H-purine-6-yl)-piperazin-1-yl]-5-fluoro-phenyl}-methanol
2-[4-(2-chloro-9H-purin-6-yl)-piperazin-1-yl]-nicotinamide
2-chloro-4-(4-pyridin-2-yl-piperazin-1-yl)-7H-pyrrolo[2,3-d]pyrimidine
5-chloro-7-(4-pyridin-2-yl-piperazin-1-yl)-3H-imidazo[4,5-b]pyridine monohydrochloride
2-[4-(5-chloro-3H-imidazo[4,5-b]pyridin-7-yl)-piperazin-1-yl]-benzamide

Thus, among the compounds corresponding to general formula (IA) or (IB), we may mention the following compounds:
2-chloro-6-[4-(phenylmethyl)-piperazin-1-yl]-1H-purine monohydrochloride
2-chloro-6-[4-(pyridin-2-yl)-piperazin-1-yl]1H-purine
2-chloro-6-(morpholin-4-yl)-1H-purine
2-chloro-6-(thiamorpholin-4-yl)-1H-purine
2-chloro-6-(piperidin-1-yl)-1H-purine 2-chloro-6-[4-(diphenylmethyl)-piperazin-1-yl]1H-purine
2-chloro-6-[4-(phenylmethyl)-piperidin-1-yl]-1H-purine monohydrochloride
2-chloro-6-(4-phenyl-piperazin-1-yl)-1H-purine monohydrochloride
2-chloro-6-[4-(pyridin-3-yl)-piperazin-1-yl]1H-purine
2-chloro-6-[4-(3-carboxamido-phenyl)-piperazin-1-yl]1H-purine
2-chloro-6-[4-(phenyl)carbonyl-piperazin-1-yl]1H-purine
2-chloro-6-[4-(phenylamino)-piperidin-1-yl]-1H-purine monohydrochloride
2-chloro-6-[4-(phenylmethyl)-piperidin-1-yl]-1H-purine monohydrochloride
2-chloro-6-[4-(6-methyl-pyridin-2-yl)-piperazin-1-yl]-1H-purine monohydrochloride
2-chloro-6-[4-(pyrimidin-2-yl)-piperazin-1-yl]-1H-purine monohydrochloride
2-chloro-6-[4-(quinolein-2-yl)-piperazin-1-yl]-1H-purine monohydrochloride
2-chloro-6-[4-(2-trifluoromethyl-phenyl)-piperazin-1-yl]-1H-purine
2-fluoro-[4-(pyridin-2-yl)-piperazin-1-yl]1H-purine

According to a second aspect, the invention relates to pharmaceutical compositions comprising a product according to its first aspect, in combination with a pharmaceutically acceptable excipient.

A product according to the invention can be used advantageously as an agent for inhibiting the activity of the Hsp90 chaperone, as an agent for inhibiting the ATPase catalytic activity of the Hsp90 chaperone, as an anti-cancer agent or for the manufacture of a medicinal product that can be used for treating a pathologic state, preferably cancer.

In general, products of general formula (IA) or (IB) according to the invention, in which A is a nitrogen atom, can be prepared by the action of a primary or 25 secondary amine on a 2,6-dihalopurine (or a 6-halopurine) according to scheme 1, in particular using the method described in J. Amer. Chem. Soc. (1959), 81, 3789-92.

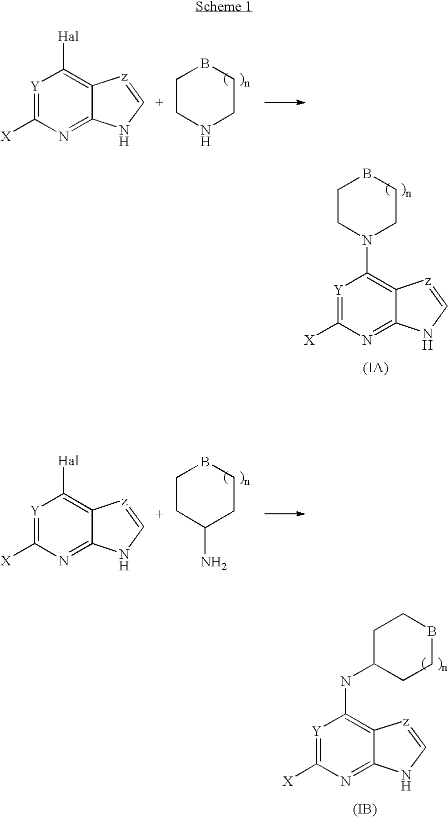

The compounds of general formula (IA) or (IB) in which A is a CH radical can be prepared by coupling, in the presence of a catalyst such as palladium tetrakis(triphenylphosphine), of an organometallic derivative of cycloalkane or of heterocycloalkane (with B=CH2, CHR, O, S, NH or NR) on a 2,6-dihalopurine (or a 6-halopurine), of which the nitrogen atom in position 7 has been protected beforehand, according to scheme 2, in particular using an organozinc compound according to the method described in Nucleoside, Nucleotide & Nucleic acids 2000, 1123.

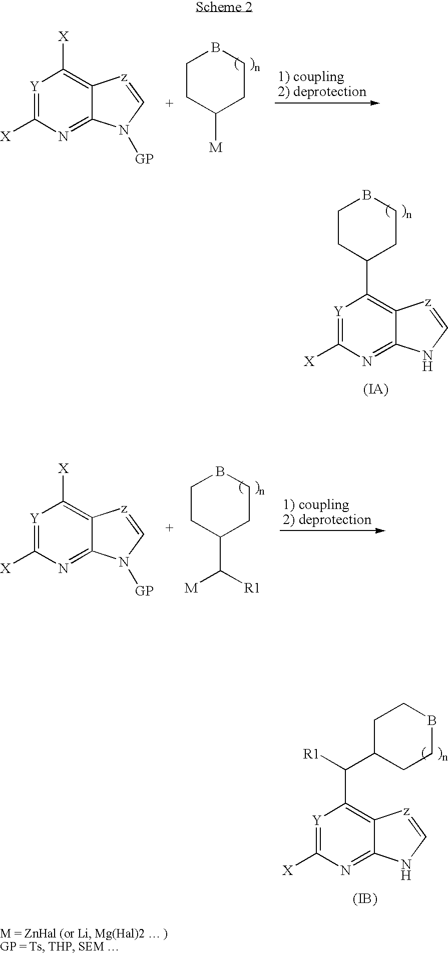

The compounds of general formula (IB) in which A is an oxygen or sulfur atom can be prepared by the action of an alcoholate or of a thioalcoholate of an alkali metal or alkaline earth metal, on a 2,6-dihalopurine (or a 6-halopurine) according to scheme 3, in particular using the method described in Tetrahedron Lett. 2001, 8161.

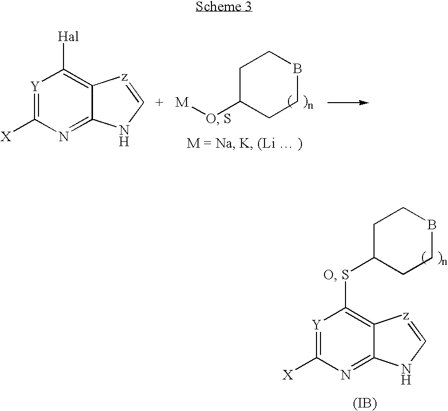

The above examples illustrate the products of the invention, but without limiting them.

### EXAMPLE 1

2-Chloro-6-[4-(phenylmethyl)-piperazin-1-yl]-1H-purine monohydrochloride

In a 50-mL flask, dissolve 500 mg of 2,6-dichloro-1H-purine in 10 mL of butanol and 1 mL of propan-2-ol then add 493 μL of 4-(phenylmethyl)piperazine and heat to 75° C. After heating for about 3 h, a white precipitate begins to appear. After heating for 5 h, reaction is complete (TLC on 60F254 silica plate—eluent dichloromethane/methanol 90/10 by volume). After cooling to 10° C., the precipitate formed is dried, and washed successively with 0.5 mL butanol, twice with 1 mL of methanol and twice with 1 mL of ethyl ether. 720 mg of 2-chloro-6-[4-(phenylmethyl)-piperazin-1-yl]-1H-purine monohydrochloride is obtained, in the form of a white powder with the following characteristics:
Melting point (Kofler)=264-6° C.
Mass spectrum (EI): m/z=328 (M+)

The Products from Examples 8 to 9 and 16 to 21:

### EXAMPLE 8

2-Chloro-6-[4-(phenylmethyl)-piperidin-1-yl]-1H-purine monohydrochloride

### EXAMPLE 9

2-Chloro-6-(4-phenyl-piperazin-1-yl)-1H-purine monohydrochloride

### EXAMPLE 16

2-Chloro-6-[4-(phenylamino)-piperidin-1-yl]-1H-purine monohydrochloride

### EXAMPLE 17

2-Chloro-6-[4-(2-chlorophenyl)-piperidin-1-yl]-1H-purine monohydrochloride

### EXAMPLE 18

2-Chloro-6-[4-(6-methyl-pyridin-2-yl)-piperazin-1-yl]-1H-purine monohydrochloride

### EXAMPLE 19

2-Chloro-6-[4-(pyrimidin-2-yl)-piperazin-1-yl]-1H-purine monohydrochloride

### EXAMPLE 20

2-Chloro-6-[4-(2-methoxy-phenyl)-piperazin-1-yl]-1H-purine monohydrochloride

### EXAMPLE 21

2-Chloro-6-[4-(quinolein-2-yl)-piperazin-1-yl]-1H-purine monohydrochloride are obtained by the procedure as in Example 1, replacing 4-(phenylmethyl)piperazine with the corresponding starting amines.

### EXAMPLE 13

2-Chloro-6-[4-(3-chloro-phenyl)-piperazin-1-yl]1H-purine

In a 50-mL flask, dissolve 400 mg of 2,6-dichloro-1H-purine in 10 mL of butanol and 1 mL of propan-2-ol, then add 458 mg of (3-chloro-phenyl)-piperazine and heat to 75° C. for 7 hours. After cooling, the precipitate formed is drained, then washed with a saturated aqueous solution of sodium bicarbonate and stove-dried at 50° C. After purification by flash-chromatography on silica gel, eluting with mixtures of dichloromethane and methanol (95-5 then 80-20 by volume), we obtain 391 mg of 2-chloro-6-[4-(3-chloro-phenyl)-piperazin-1-yl]1H-purine, in the form of a white powder with the following characteristics:
Melting point (Kofler)>260° C.
Mass spectrum (EI): m/z=349 (M+)

The products from Examples 2 to 7, 10 to 12, 14 to 15 and 22 are obtained by the procedure as in Example 13, replacing (3-chloro-phenyl)-piperazine with the corresponding starting amines or with sodium phenylmethanolate:

### EXAMPLE 2

2-chloro-6-[4-(pyridin-2-yl)-piperazin-1-yl]1H-purine

### EXAMPLE 3

2-chloro-6-(morpholin-4-yl)-1H-purine

### EXAMPLE 4

2-chloro-6-(thiamorpholin-4-yl)-1H-purine

### EXAMPLE 5

2-chloro-6-(piperidin-1-yl)-1H-purine

### EXAMPLE 6

2-chloro-6-[4-(diphenylmethyl)-piperazin-1-yl]1H-purine

### EXAMPLE 7

2-chloro-6-cyclopentylamino-1H-purine

### EXAMPLE 10

2-chloro-6-[4-(3,4-dimethoxy-phenyl)methyl-piperazin-1-yl]1H-purine

### EXAMPLE 11

2-chloro-6-[4-(pyridin-3-yl)-piperazin-1-yl]1H-purine

### EXAMPLE 12

2-chloro-6-[4-(3-carboxamido-phenyl)-piperazin-1-yl]1H-purine

### EXAMPLE 14

2-chloro-6-[4-(3,4,5-trimethoxy-phenyl)carbonyl-piperazin-1-yl]1H-purine

### EXAMPLE 15

2-chloro-6-[4-(phenyl)carbonyl-piperazin-1-yl]1H-purine

### EXAMPLE 22

2-chloro-6-[4-(2-trifluoromethyl-phenyl)-piperazin-1-yl]1H-purine

### EXAMPLE 23

2-Fluoro-[4-(pyridin-2-yl)-piperazin-1-yl]1H-purine

Stage 1: Pour 15 mL of a 65% solution of hydrofluoric acid in pyridine into a 25-mL three-necked flask under an argon atmosphere, cool to −50° C., then add, portion by portion, 1 g of 2-amino-6-chloropurine, which can be obtained according to Helv. Chim. Acta 1986, 69, 1602-13. Then pour in slowly, in 10 minutes at a temperature between −60° and −50° C., 1 mL of tert.butyl nitrite. After stirring for a further 30 minutes, stop the reaction (LC/MS analysis). Pour the reaction mixture slowly into a 30% aqueous solution of sodium hydroxide. After concentrating under reduced pressure, the yellow residue obtained is purified by flash-chromatography on 100 g of silica gel (70-230 mesh), eluting with a mixture of dichloromethane and methanol (90/10 by volume). On concentrating the fractions eluted between 400 and 500 mL, we obtain 360 mg of 6-chloro-2-fluoropurine in the form of a white solid, which is used “as is” in the next stage.

Stage 2: Heat overnight, at 90° C., a solution of 200 mg of 2-fluoro-6-chloropurine and 1-(pyridin-2-yl)piperazine in 6 mL of n-butanol. After cooling, add 5 mL of a 28% aqueous ammonia solution and concentrate to dryness under reduced pressure. The residue is purified by flash-chromatography on silica gel (40-60 μM), eluting with a mixture of dichloromethane and methanol (98-2 by volume). We thus obtain 38 mg of 2-fluoro-[4-(pyridin-2-yl)-piperazin-1-yl]1H-purine, in the form of a white solid with the following characteristics:
melting point (Kofler)>260° C.
mass spectrum (E/I): m/z=299 (M+)

The products from Examples 24, 26 and 27 are obtained by the procedure as in Example 13, replacing the (3-chloro-phenyl)-piperazine with the corresponding starting amines:

### EXAMPLE 24

2-Chloro-6-[4-(3-trifluoromethyl-pyridin-2-yl)-piperazin-1-yl]-9H-purine.

### EXAMPLE 26

{2-[4-(2-Chloro-9H-purin-6-yl)-piperazin-1-yl]-5-fluoro-phenyl}-methanol.

### EXAMPLE 27

2-[4-(2-Chloro-9H-purin-6-yl)-piperazin-1-yl]-nicotinamide.

### EXAMPLE 25

2-[4-(5-Chloro-3H-imidazo[4,5-b]pyridin-7-yl)-piperazin-1-yl]-benzamide.

Heat, for 6 hours at 80° C., a solution of 230 mg of 2-[4-(5-chloro-3H-imidazo[4,5-b]pyridin-7-yl)-piperazin-1-yl]-benzonitrile in 2 mL of tert-butanol containing 87.7 mg of potassium hydroxide. After concentrating under reduced pressure, the reaction mixture is taken up in 50 mL of water and extracted 3 times with 25 mL of dichloromethane. The organic phases are combined, washed with water, dried over magnesium sulfate and concentrated to dryness under reduced pressure. After purification by flash-chromatography on silica gel (70-230 mesh), eluting with a mixture of dichloromethane and methanol (95/5 by volume), we obtain 65 mg of 2-[4-(5-chloro-3H-imidazo[4,5-b]pyridin-7-yl)-piperazin-1-yl]-benzamide, in the form of a white meringue with the following characteristics:
melting point (Kofler)>260° C.
mass spectrum (E/I): m/z=357 (M+)

2-[4-(5-Chloro-3H-imidazo[4,5-b]pyridin-7-yl)-piperazin-1-yl]-benzonitrile can be obtained as in Example 13, replacing the (3-chloro-phenyl)-piperazine with (2-cyano-phenyl)-piperazine.

### EXAMPLE 28

4-(4-Phenylmethyl-piperazin-1-yl)-2-chloro-7H-pyrrolo[2,3-d]pyrimidine.

Follow the procedure in Example 1, but starting from 450 mg of 2,4-dichloro-7H-pyrrolo[2,3-d]pyrimidine and 50 mg of 1-phenylmethyl-piperazine heated at 80° C. for 7 hours in 2 mL of n-butanol. After filtration of the precipitate formed, we thus obtain 36 mg of 4-(4-phenylmethyl-piperazin-1-yl)-2-chloro-7H-pyrrolo[2,3-d]pyrimidine, in the form of pale yellow crystals with the following characteristics:
melting point (Kofler)=250° C.
mass spectrum (E/I): m/z=327 (M+).

2,4-Dichloro-7H-pyrrolo[2,3-d]pyrimidine can be obtained according to J. Med. Chem. 1988, 31(8), 1501-6.

### EXAMPLE 29

2-Chloro-4-(4-pyridin-2-yl-piperazin-1-yl)-7H-pyrrolo[2,3-d]pyrimidine.

Follow the procedure in Example 13, starting from 90 mg of 2,4-dichloro-7H-pyrrolo[2,3-d]pyrimidine and 82 mg of 1-(2-pyrdidyl)piperazine at 85° C. for 20 hours. After purification by flash-chromatography on silica gel (70-230 mesh), eluting with a mixture of dichloromethane and methanol (95/5 by volume), we obtain 15 mg of 2-chloro-4-(4-pyridin-2-yl-piperazin-1-yl)-7H-pyrrolo[2,3-d]pyrimidine, in the form of a light beige solid with the following characteristics:
mass spectrum (E/I): m/z=314 (M+).

### EXAMPLE 30

5-Chloro-7-(4-pyridin-2-yl-piperazin-1-yl)-3H-imidazo[4,5-b]pyridine monohydrochloride.

Follow the procedure in Example 13, starting from 375 mg of 5,7-dichloro-1H-imidazo[4,5-b]pyridine and 163 mg of 1-(2-pyrdidyl)piperazine at 95° C. for 40 hours. After purification by flash-chromatography on silica gel (70-230 mesh), eluting with a mixture of dichloromethane and methanol (95/5 by volume), then crystallization from a 1 M solution of hydrochloric acid in isopropanol, we obtain 35 mg of 5-chloro-7-(4-pyridin-2-yl-piperazin-1-yl)-3H-imidazo[4,5-b]pyridine monohydrochloride, in the form of fine white crystals with the following characteristics:
melting point (Kofler)>260° C.
mass spectrum (E/I): m/z=351 (M+).

5,7-Dichloro-1H-imidazo[4,5-b]pyridine can be obtained by the procedure according to Bioorg. Med. Chem. 2003, 11(6), 899-908.

### EXAMPLE 31

2-[4-(5-Chloro-3H-imidazo[4,5-b]pyridin-7-yl)-piperazin-1-yl]-benzamide

Following the procedure in Example 25, but starting from 300 mg of 2-[4-(5-chloro-3H-imidazo[4,5-b]pyridin-7-yl)-piperazin-1-yl]-benzonitrile and 127 mg of potassium hydroxide in 4 mL of tert-butanol, we obtain 120 mg of 2-[4-(5-chloro-3H-imidazo[4,5-b]pyridin-7-yl)-piperazin-1-yl]-benzamide, in the form of an amorphous white solid with the following characteristics:
mass spectrum (E/I): m/z=356 (M+).

2-[4-(5-Chloro-3H-imidazo[4,5-b]pyridin-7-yl)-piperazin-1-yl]-benzonitrile can be obtained as in Example 30, replacing the 1-(2-pyrdidyl)piperazine with (2-cyanophenyl)-piperazine.

Biological test for biological characterization of the invention:

The inorganic phosphate released during the hydrolysis of ATP by the ATPase activity of Hsp82 is quantified by the malachite green method. In the presence of this reagent, there is formation of the inorganic phosphate-molybdate-malachite green complex, which absorbs at a wavelength of 620 nm.

The products to be evaluated are incubated in a reaction volume of 30 μl, in the presence of 1 μM Hsp82 and 250 μM of substrate (ATP) in a compound buffer of 50 mM Hepes-NaOH (pH 7.5), 1 mM DTT, 5 mM MgCl2 and 50 mM KCl at 37° C. for 60 min. In parallel, a gradient of inorganic phosphate from 1 to 40 μM is prepared in the same buffer. The ATPase activity is then developed by adding 60 μl of the reagent biomol green (Tebu). After incubating for 20 min at room temperature, the absorbance of the different wells is measured using a microplate reader at 620 nm. The concentration of inorganic phosphate of each sample is then calculated from the calibration curve. The ATPase activity of Hsp82 is expressed as concentration of inorganic phosphate produced in 60 min. The effect of the various products tested is expressed as percentage inhibition of the ATPase activity.

The formation of ADP due to the ATPase activity of Hsp82 was utilized for developing another method for evaluating the enzymatic activity of this enzyme by the application of an enzymatic coupling system employing pyruvate kinase (PK) and lactate dehydrogenase (LDH). In this spectrophotometric method of the kinetic type, the PK catalyzes the formation of ATP and pyruvate from phosphenol-pyruvate (PEP) and of ADP produced by HSP82. The pyruvate formed, which is a substrate of LDH, is then converted to lactate in the presence of NADH. In this case, the decrease in the concentration of NADH, measured from the decrease in absorbance at a wavelength of 340 nm, is proportional to the concentration of ADP produced by HSP82.

The test products are incubated in a reaction volume of 100 μl of compound buffer of 100 mM Hepes-NaOH (pH 7.5), 5 mM MgCl2, 1 mM DTT, 150 mM KCl, 0.3 mM NADH, 2.5 mM PEP and 250 μM ATP. This mixture is preincubated at 37° C. for 30 min before adding 3.77 units of LDH and 3.77 units of PK. The reaction is initiated by addition of the product to be evaluated, at variable concentrations, and of Hsp82, at a concentration of 1 μM. Then the enzymatic activity of Hsp82 is measured, continuously, in a microplate reader, at 37° C., at a wavelength of 340 nm. The initial rate of the reaction is obtained by measuring the slope of the tangent of the recorded curve to the origin. The enzymatic activity is expressed as μM of ADP formed per minute. The effect of the various products tested is expressed as percentage inhibition of the ATPase activity.

The inhibitory activities on the ATPase activity of Hsp82 obtained with the products of the invention in the enzymatic coupling system are presented in the following table, according to the following criteria of inhibition of the ATPase activity of Hsp82:

| Example | Structure | IC50 μM |
|---|---|---|
| 1 | 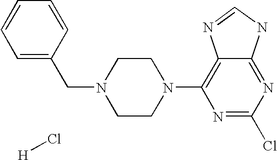 | A |
| 2 | 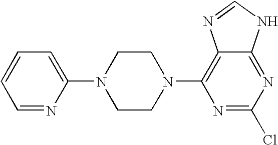 | A |
| 3 | 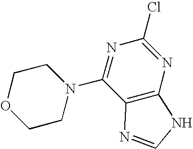 | B |
| 4 | 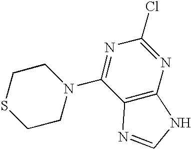 | B |
| 5 | 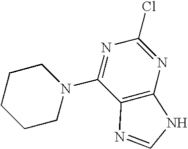 | A |
| 6 | 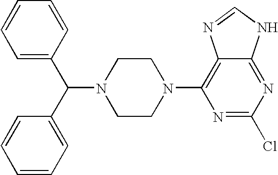 | B |
| 7 | 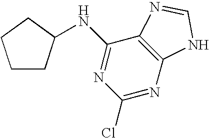 | C |
| 8 | 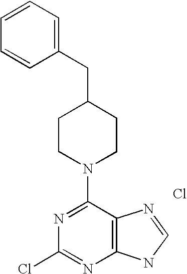 | A |
| 9 | 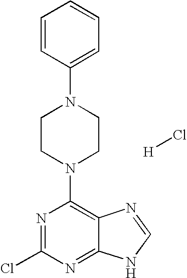 | B |
| 10 | 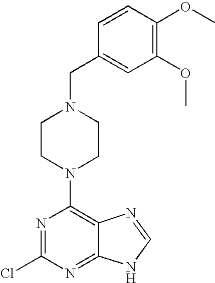 | C |
| 11 | 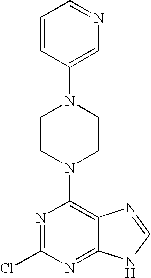 | B |
| 12 | 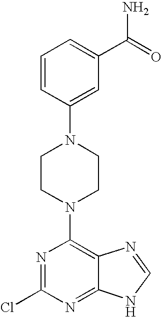 | B |
| 13 | 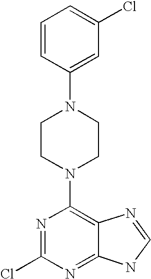 | C |
| 14 | 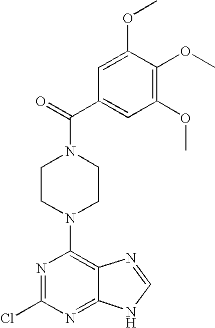 | C |
| 15 | 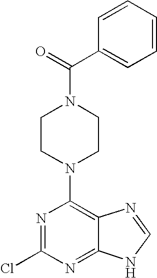 | B |
| 16 | 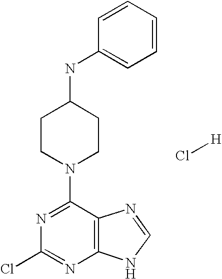 | A |
| 17 | 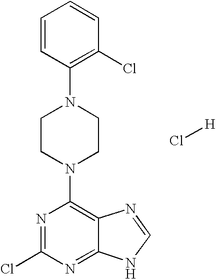 | C |
| 18 | 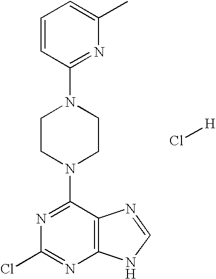 | B |
| 19 | 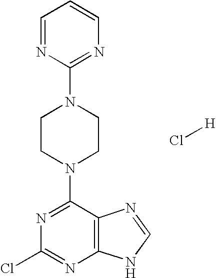 | A |
| 20 | 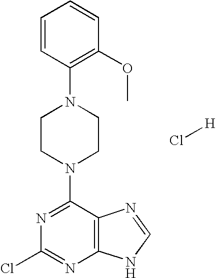 | C |
| 21 | 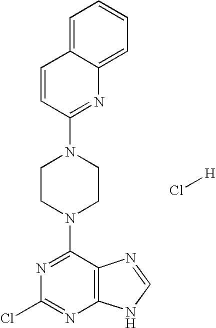 | A |
| 22 | 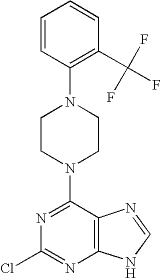 | B |
| 23 | 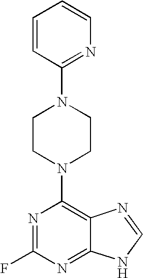 | B |
| 24 | 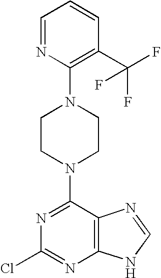 | B |
| 25 | 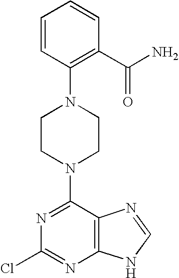 | A |
| 26 | 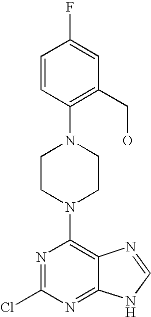 | A |
| 27 | 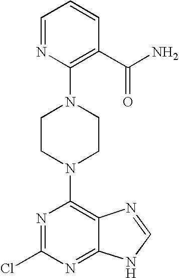 | A |
| 28 | 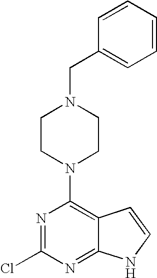 | B |
| 29 | 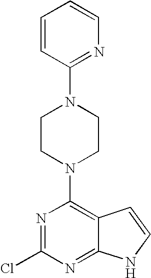 | A |
| 30 | 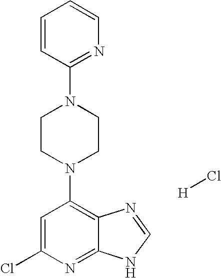 | A |
| 31 | 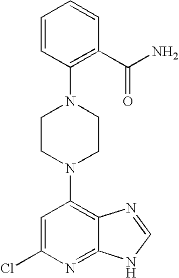 | A |

A: IC50 < 1 μM
B: 1 μM < IC50 < 10 μM
C: 10 μM < IC50 < 100 μM

What is claimed is:

1. A compound of formula (IA1) 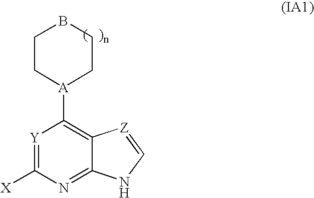
wherein:
Y and Z, which may be identical or different, represent N or CH, provided that at least one of Y and Z represents N;
X represents a halogen atom;
A represents N or CH;
B represents O, S, NR′, CH2 or CHR′;
R′ represents a hydrogen atom; or a C1-C7 alkyl radical; or a C2-C7 alkenyl or alkynyl radical; or a (CH2)ₙ-aryl or heteroaryl radical; a —CH(aryl)₂ radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, or
O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer;
provided that the compound of formula (IA1) is other than

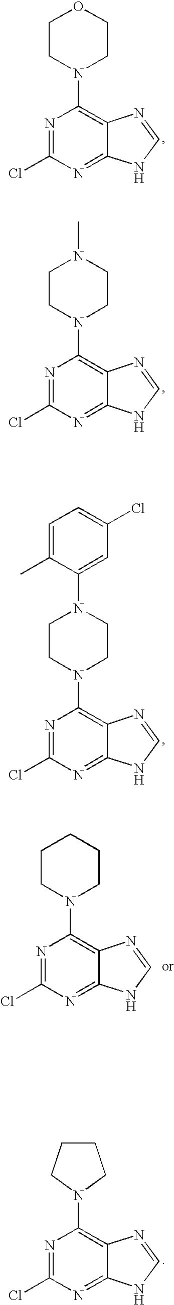

2. A compound of formula (IB1) 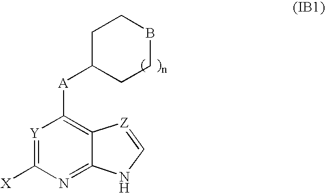
wherein:
Y and Z, which may be identical or different, represent N or CH, provided that at least one of Y and Z represents N;
X represents a halogen atom;
A O, S, NH, CH2 or CHR;
B represents O, S, NR′, CH2 or CHR′;
R represents a hydrogen atom, or a C1-C3 alkyl radical;
R′ represents a hydrogen atom; or a C1-C7 alkyl radical; or a C2-C7 alkenyl or alkynyl radical; or a (CH2)ₙ-aryl or heteroaryl radical; a —CH(aryl)₂ radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and
O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer;
provided that the compound of formula (IB1) is other than

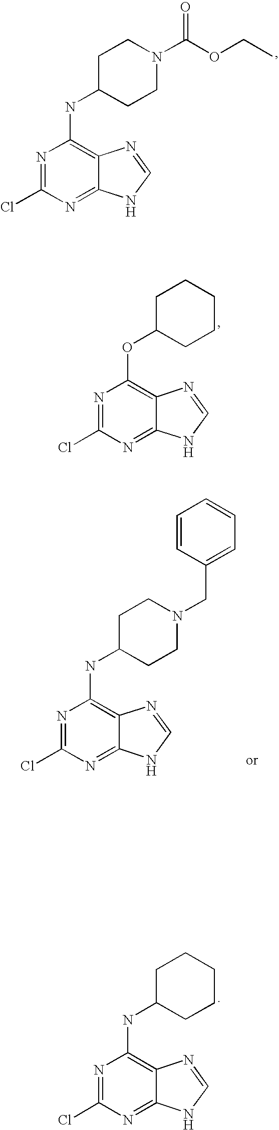

3. A compound according to claim 1 of formula (IA), (IIA) or (IIIA) 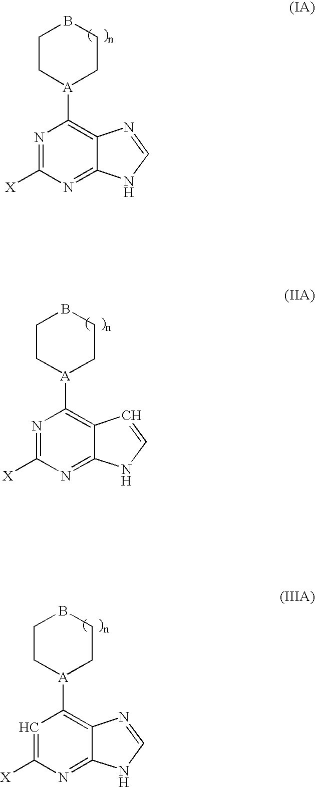
wherein:
X represents a halogen atom;
A represents N or CH;
B represents O, S, NR′, CH2 or CHR′;
R′ represents a hydrogen atom; or a C1-C7 alkyl radical; or a C2-C7 alkenyl or alkynyl radical; or a (CH2)ₙ-aryl or heteroaryl radical; a —CH(aryl)₂ radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

4. A compound according to claim 2, of formula (IB), (IIB) or (IIIB) 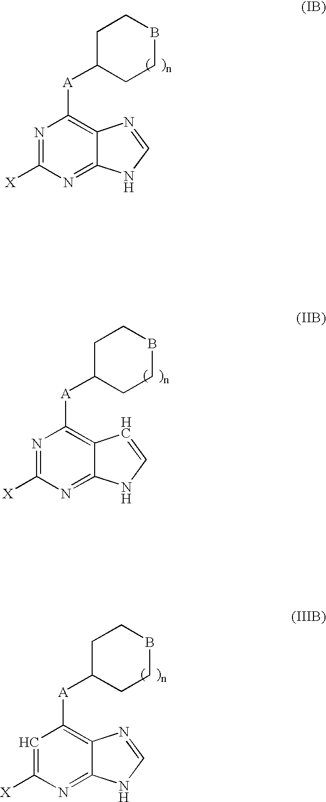
wherein:
X represents a halogen atom;
A represents O, S, NH, CH2 or CHR;
B represents O, S, NR′, CH2 or CHR′;
R represents a hydrogen atom; or a C1-C3 alkyl radical;
R′ represents a hydrogen atom; or a C1-C7 alkyl radical; or a C2-C7 alkenyl or alkynyl radical; or a (CH2)ₙ-aryl or heteroaryl radical; a —CH(aryl)₂ radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3; and
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

5. A compound according to claim 1 of formula (IA) 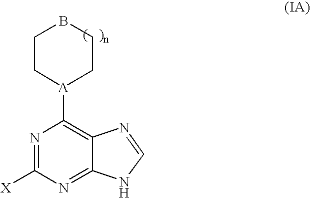
wherein:
X represents a halogen atom;
A represents N or CH;
B represents O, S, NR′, CH2 or CHR′;
R′ represents a hydrogen atom; or a C1-C7 alkyl radical; or a C2-C7 alkenyl or alkynyl radical; or a (CH2)ₙ-aryl or heteroaryl radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

6. A compound according to claim 2 of formula (1B) 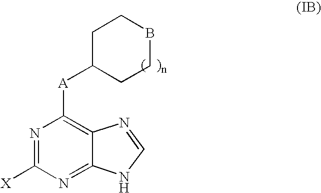
wherein:
X represents a halogen atom;
A represents O, S, NH, CH2 or CHR;
B represents O, S, NR′, CH2 or CHR′;
R represents a hydrogen atom; or a C1-C3 alkyl radical;
R′ represents a hydrogen atom; or a C1-C7 alkyl radical; or a C2-C7 alkenyl or alkynyl radical; or a (CH2)ₙ-aryl or heteroaryl radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, hydroxyalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

7. A compound according to claim 1 of formula (IA) 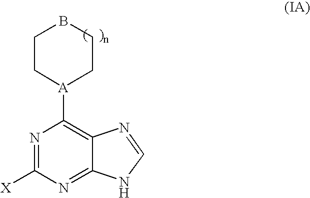
wherein:
X represents a halogen atom;
A represents N or CH;
B represents O, S, NR′, CH2 or CHR′;
R′ represents a hydrogen atom; or a C1-C7 alkyl radical; or a C2-C7 alkenyl or alkynyl radical; or a (CH2)ₙ-aryl or heteroaryl radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

8. A compound according to claim 2 of formula (IB) 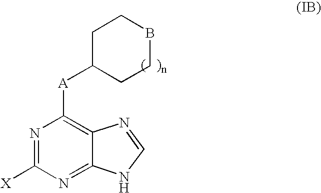
wherein:
X represents a halogen atom;
A represents O, S, NH, CH2 or CHR;
B represents O, S, NR′, CH2 or CHR′;
R represents a hydrogen atom; or a C1-C3 alkyl radical
R′ represents a hydrogen atom; or a C1-C7 alkyl radical; or a C2-C7alkenyl or alkynyl radical; or a (CH2)ₙ-aryl or heteroaryl radical; or a C(Z1)-aryl or heteroaryl radical; where the aryl or heteroaryl rings, which may be mono- or bicyclic with 5 to 10 ring members, can contain from 0 to 3 heteroatoms, which may be identical or different, selected from O, S or N, and can optionally be substituted with one or more radicals, which may be identical or different, selected from halogen atoms, alkyl, OH, Oalkyl, SH, Salkyl, NH₂, NHalkyl, N(alkyl)₂, CF₃, CN, NO₂, COOH, C(O)Oalkyl, CONH₂, C(O)NHalkyl, C(O)N(alkyl)₂, S(O)alkyl, S(O)₂alkyl, SONH₂, S(O)₂NH₂, S(O)₂NHalkyl, S(O)₂N(alkyl)₂, —C(O)NH₂, P(O)(OH)₂, P(O)(alkyl)OH, P(O)(Oalkyl)₂, P(O)(alkyl)Oalkyl, NH—C(O)—NH₂, NH(CO)NHalkyl, NH(CO)N(alkyl)₂, O—C(O)NHalkyl, and O—C(O)N(alkyl)₂, the alkyl portions of which can be C1-C3;
n=0, 1, 2; and
Z1 represents an oxygen or sulfur atom or a radical NR′; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

9. A compound according to claim 1 wherein X is Cl.

10. A compound according to claim 2 wherein X is Cl.

11. A compound according to claim 1 wherein A is N.

12. A compound according to claim 9 wherein B is NR′ or CHR′.

13. A compound according to claim 10 wherein B is NR′ or CHR′.

14. A compound according to claim 11 wherein B is NR′ or CHR′.

15. A compound according to claim 1 wherein n is 1.

16. A compound according to claim 2 wherein n is 1.

17. A compound according to claim 12 wherein n is 1.

18. A compound according to claim 13 wherein n is 1.

19. A compound according to claim 14 wherein n is 1.

20. A compound according to claim 1 wherein R′ is selected from the group consisting of phenyl, phenylmethyl, phenylamino, phenylcarbonyl, pyridyl, pyrimidinyl and quinoleinyl, the phenyl and pyridyl radicals being optionally substituted with one or more radicals, which may be identical or different, selected from halogen atoms, alkyl, hydroxyalkyl, Oalkyl, CF3 and CONH2 radicals.

21. A compound according to claim 2 wherein R′ is selected from the group consisting of phenyl, phenylmethyl, phenylamino, phenylcarbonyl, pyridyl, pyrimidinyl and quinoleinyl, the phenyl and pyridyl radicals being optionally substituted with one or more radicals, which may be identical or different, selected from halogen atoms, alkyl, hydroxyalkyl, Oalkyl, CF3 and CONH2 radicals.

22. A compound according to claim 20 wherein R′ is phenyl, or phenyl substituted with at least one halogen atom, Oalkyl, or —C(O)NH2, or R′ is phenylmethyl, phenylamino, pyridyl, pyrimidinyl or quinoleinyl.

23. A compound according to claim 21 wherein R′ is phenyl, or phenyl substituted with at least one halogen atom, Oalkyl, or —C(O)NH2, or R′ is phenylmethyl, phenylamino, pyridyl, pyrimidinyl or quinoleinyl.

24. A compound according to claim 2 wherein A is NH.

25. A compound according to claim 9 wherein B is CH2.

26. A compound according to claim 24 wherein B is CH2.

27. A compound according to claim 24 wherein n is 0.

28. A compound according to claim 25 wherein n is 0.

29. A compound according to claim 26 wherein n is 0.

30. A compound according to claim 1 which is
2-chloro-6-[4-(phenylmethyl)-piperazin-1-yl]-1H-purine;
2-chloro-6-[4-(pyridin-2-yl)-piperazin-1-yl]1H-purine;
2-chloro-6-(morpholin-4-yl)-1H-purine;
2-chloro-6-(thiamorpholin-4-yl)-1H-purine;
2-chloro-6-(piperidin-1-yl)-1H-purine;
2-chloro-6-[4-(diphenylmethyl)-piperazin-1-yl]1H-purine;
2-chloro-6-[4-(phenylmethyl)-piperidin-1-yl]-1H-purine;
2-chloro-6-(4-phenyl-piperazin-1-yl)-1H-purine;
2-chloro-6-[4-(pyridin-3-yl)-piperazin-1-yl]1H-purine;
2-chloro-6-[4-(3-carboxamido-phenyl)-piperazin-1-yl]1H-purine;
2-chloro-6-[4-(phenyl)carbonyl-piperazin-1-yl]1H-purine;
2-chloro-6-[4-(phenylamino)-piperidin-1-yl]-1H-purine;
2-chloro-6-[4-(phenylmethyl)-piperidin-1-yl]-1H-purine;
2-chloro-6-[4-(6-methyl-pyridin-2-yl)-piperazin-1-yl]-1H-purine;
2-chloro-6-[4-(pyrimidin-2-yl)-piperazin-1-yl]-1H-purine;
2-chloro-6-[4-(quinolein-2-yl)-piperazin-1-yl]-1H-purine;
2-chloro-6-[4-(2-trifluoromethyl-phenyl)-piperazin-1-yl]1H-purine;
2-fluoro-[4-(pyridin-2-yl)-piperazin-1-yl]1H-purine;
2-[4-(5-chloro-3H-imidazo[4,5-b]pyridin-7-yl)-piperazin-1-yl]-benzamide;
{2-[4-(2-chloro-9H-purin-6-yl)-piperazin-1-yl]-5-fluoro-phenyl}-methanol;
2-[4-(2-chloro-9H-purin-6-yl)-piperazin-1-yl]-nicotinamide;
2-chloro-4-(4-pyridin-2-yl-piperazin-1-yl)-7H-pyrrolo[2,3-d]pyrimidine;
5-chloro-7-(4-pyridin-2-yl-piperazin-1-yl)-3H-imidazo[4,5-b]pyridine; or
2-[4-(5-chloro-3H-imidazo[4,5-b]pyridin-7-yl)-piperazin-1-yl]-benzamide; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

31. A compound according to claim 1 which is
2-chloro-6-[4-(phenylmethyl)-piperazin-1-yl]-1H-purine;
2-chloro-6-[4-(pyridin-2-yl)-piperazin-1-yl]1H-purine;
2-chloro-6-(morpholin-4-yl)-1H-purine;
2-chloro-6-(thiamorpholin-4-yl)-1H-purine;
2-chloro-6-(piperidin-1-yl)-1H-purine;
2-chloro-6-[4-(diphenylmethyl)-piperazin-1-yl]1H-purine;
2-chloro-6-[4-(phenylmethyl)-piperidin-1-yl]-1H-purine;
2-chloro-6-(4-phenyl-piperazin-1-yl)-1H-purine;
2-chloro-6-[4-(pyridin-3-yl)-piperazin-1-yl]1H-purine;
2-chloro-6-[4-(3-carboxamido-phenyl)-piperazin-1-yl]1H-purine;
2-chloro-6-[4-(phenyl)carbonyl-piperazin-1-yl]1H-purine;
2-chloro-6-[4-(phenylamino)-piperidin-1-yl]-1H-purine;
2-chloro-6-[4-(phenylmethyl)-piperidin-1-yl]-1H-purine;
2-chloro-6-[4-(6-methyl-pyridin-2-yl)-piperazin-1-yl]-1H-purine;
2-chloro-6-[4-(pyrimidin-2-yl)-piperazin-1-yl]-1H-purine;
2-chloro-6-[4-(quinolein-2-yl)-piperazin-1-yl]-1H-purine;
2-chloro-6-[4-(2-trifluoromethyl-phenyl)-piperazin-1-yl]1H-purine;
2-fluoro-[4-(pyridin-2-yl)-piperazin-1-yl]1H-purine; or
a tautomer, racemate, enantiomer or diastereomer of said compound, or a pharmaceutically acceptable mineral acid or organic acid addition salt, or pharmaceutically acceptable inorganic or organic base addition salt of said compound, tautomer, racemate, enantiomer or diastereomer.

32. A compound according to claim 2 which is 2-chloro-6-cyclopentylamino-1H-purine or a tautomer or diastereomer of said compound, or a pharmaceutically acceptable mineral acid or organic acid addition salt, or pharmaceutically acceptable inorganic or organic base addition salt of said compound, tautomer or diastereomer.

33. A pharmaceutical composition comprising a compound according to claim 1 in combination with a pharmaceutically acceptable excipient.

34. A pharmaceutical composition comprising a compound according to claim 2 in combination with a pharmaceutically acceptable excipient.

35. The use of a compound according to claim 1 as an agent for inhibiting the activity of the Hsp90 chaperone.

36. The use of a compound according to claim 2 as an agent for inhibiting the activity of the Hsp90 chaperone.

37. A method for inhibiting the Hsp90 chaperone, in a patient in need of such inhibition, comprising administering to said patient a pharmaceutically effective amount of a compound according to claim 1.

38. A method for inhibiting the Hsp90 chaperone, in a patient in need of such inhibition, comprising administering to said patient a pharmaceutically effective amount of a compound according to claim 2.

39. A method of treating cancer, in a patient in need of such treatment, comprising administering to such patient a pharmaceutically effective amount of a compound according to claim 1.

40. A method of treating cancer, in a patient in need of such treatment, comprising administering to such patient a pharmaceutically effective amount of a compound according to claim 2.
